(19) United States
(12) Patent Application Publication
(10) Pub. No.: US 2010/0280595 A1
(43) Pub. Date: Nov. 4, 2010

(54) Method and Device for Localized Administration of Calcium Chelating Agent

(75) Inventors: Iskender Bilge, Charlottesville, VA (US); Joseph Berglund, Santa Rosa, CA (US); Ayala Hezi-Yamit, Windsor, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/769,803

(22) Filed: Apr. 29, 2010

Related U.S. Application Data
(60) Provisional application No. 61/174,165, filed on Apr. 30, 2009.

(51) Int. Cl. A61L 27/54; A61M 25/00; A61F 2/82; A61M 25/10; A61F 2/84

(52) U.S. Cl. 623/1.23; 604/508; 623/1.1; 604/509; 623/1.24; 604/523

(57) ABSTRACT

A system for treating atherosclerotic cardiovascular disease and cardiac valve dysfunction comprises delivering one or more calcium chelating agents locally to a treatment site. One aspect of the invention provides a method including delivering a nano-particulate calcium chelating agent into a vascular wall. Another aspect of the invention provides a method and device for forming a sealed chamber around a cardiac valve, releasing one or more calcium chelating agents into the sealed chamber and decalcifying a cardiac valve in need thereof.

### RELATED APPLICATIONS

This application claims the benefit of U.S. Provisional Patent Application Ser. 61/174,165 filed Apr. 30, 2009. The disclosures of which are herein incorporated by reference in their entirety.

### TECHNICAL FIELD

This invention relates generally to methods and devices for treating atherosclerotic cardiovascular disease and cardiac valve dysfunction. More specifically, the invention relates to localized delivery of one or more calcium chelating agents within the cardiovascular system in order to remove calcium from an atherosclerotic plaque lesion or a cardiac valve.

### BACKGROUND OF THE INVENTION

Atherosclerosis, a major cause of morbidity and mortality in the United States, is a progressive disease that results in the deposition of plaque on the inner lining of large and medium-sized arteries. The plaque, consisting of fatty substances including cholesterol, cellular debris and calcium, builds up slowly, and causes clinical symptoms most often beginning in middle age. The plaque may grow large enough to partially block the artery and significantly reduce blood flow to the heart and other vital organs. If blood flow to the heart is sufficiently reduced, angina (chest pain) results.

Two types of arterial calcification have been observed widely in the adult populations of Western countries: medial arterial calcification and the calcification associated with atherosclerotic plaque. Calcification in the coronary arteries, however, almost invariably coincides with plaque formation. Although the etiology of plaque formation is not well understood, various causal factors have been identified, including high serum cholesterol concentration, hypertension, obesity, exposure to cigarette smoke or other pollutants, and the presence of concomitant disease such as diabetes. The sensitivity of an individual to each of these factors is thought to be determined at least in part by genetic heredity.

Throughout the life of the individual, the blood vessel wall is exposed to cholesterol transported in low-density lipoprotein particles. Some of the particles enter the vessel wall and release cholesterol, which is then oxidized and initiates the inflammatory process by attracting macrophage to the site. The macrophage ingest the oxidized cholesterol and become foam cells. The foam cells and platelets that accumulate at the site continue the inflammatory process, eventually leading to the destruction of smooth muscle cells and replacing them with collagen. The collagen layer eventually extends over the fatty deposit and forms a fibrous cap between the fatty deposit and the intimal lining of the vessel. Fibromuscular proliferation occurs in the vessel wall at the site of plaque formation. As the intima thickens, calcium is deposited around the base of the plaque causing the plaque to harden. Over time the artery enlarges to accommodate the growing plaque and maintain the size of the lumen. However, in some cases, the lumen of the artery eventually becomes partially blocked resulting in stenosis and reduced blood flow. In severe cases, chronic total occlusion (CTO) of the vessel may occur as a result of calcification of the growing plaque.

To treat stenosis and prevent restenosis, stents and stent grafts are frequently used at the site arterial blockages. Using a balloon catheter, the plaque is compressed against the vessel wall, and a stent is placed across the lesion, and laterally expanded so that the stent engages the vessel wall (angioplasty procedure) and maintains the diameter of the vessel lumen. Calcified plaque lesions are hard and must be cracked in order for the lesion to be more readily compressed against the vessel wall. Plaque calcification frequently causes complications such as difficulty in accessing a lesion site, difficulty positioning a stent, and trauma to the vessel wall during cracking of the lesion.

Heart valves, such as the mitral, tricuspid, aortic and pulmonic valves, are sometimes damaged by disease or by aging, resulting in problems with the proper functioning of the valve. Heart valve problems generally take one of two forms: stenosis, in which a valve does not open completely or the opening is too small, resulting in restricted blood flow, or insufficiency, in which blood leaks backward across a valve when it should be closed. Treatment involves restoring the valve to normal function by surgically removing damaged or malformed tissue and reconstructing the damaged valve. In severe cases, however, valve replacement is required to restore cardiac function.

Replacement valves are either bioprosthetic, made of animal tissue, or mechanical. In either case, the replacement valve sometimes becomes calcified, causing recurrence of valvular sclerosis, stenosis or insufficiency, and a need for additional treatment.

It is desirable, therefore, to facilitate the treatment of cardiovascular disease by providing methods and devices to remove calcium deposits from cardiovascular plaque and cardiac valves.

### SUMMARY OF THE INVENTION

One aspect of the invention provides a method for treating mineralized cardiovascular atherosclerotic plaque lesions. The method comprises delivering a microparticulate calcium chelating agent into the vascular wall at a treatment site within the cardiovascular system. The method further comprises releasing a therapeutically effective amount of the calcium chelating agent to the treatment site for a predetermined period of time and removing calcium from the mineralized portions of the plaque. Decalcification of the plaque causes the plaque to become pliable.

Another aspect of the invention provides a device for treating a calcified heart valve comprising a tubular graft member having a valve member attached to the luminal surface of the graft member. When the tubular graft is placed at the treatment site, the outer surface of the graft member forms a sealed chamber adjacent to the vessel wall at the treatment site.

Another aspect of the invention provides a method of treating a calcified heart valve comprising delivering a tubular graft to a treatment adjacent to the calcified heart valve using a catheter. The tubular graft is placed through the calcified heart valve and expanded against the vessel wall forming a sealed chamber that encloses the calcified valve. The method further comprises releasing one or more chelating agents into the sealed chamber and thereby removing calcium from the heart valve.

The invention is illustrated by the accompanying figures portraying various embodiments and the detailed description given below. The figures should not be taken to limit the invention to the specific embodiments, but are for explanation and understanding. The detailed description and figures are merely illustrative of the invention rather than limiting, the scope of the invention being defined by the appended claims and equivalents thereof. The drawings are not to scale. The foregoing aspects and other attendant advantages of the invention will become more readily appreciated by the detailed description taken in conjunction with the accompanying figures.

### BRIEF DESCRIPTION OF THE DRAWINGS

FIG. 1 is a flow chart illustrating a method for treating a calcium rich vascular lesion, in accordance with one embodiment of the invention;

FIGS. 1A-1C are schematic illustrations of the method for treating a calcium rich vascular lesion detailed in the flow chart of FIG. 1;

FIG. 2 is a schematic illustration of a dual balloon device for delivering a calcium chelating agent locally to a calcium-rich vascular lesion, in accordance with the invention;

FIG. 3 is a schematic illustration of a compliant delivery device for conforming to the geometry of a vessel and delivering a calcium chelating agent locally to a calcium-rich vascular lesion, in accordance with the invention;

FIG. 4 is a schematic illustration of a dual balloon device for delivering a calcium chelating agent locally and for withdrawing the calcium chelating agent at the end of the treatment, in accordance with the invention;

FIG. 5 is an illustration of a device for delivering a particulate calcium chelating agent into a vessel wall adjacent to a calcium rich vascular lesion, in accordance with the invention; and

FIG. 6 is a schematic illustration of a device for de-mineralizing a calcified heart valve, in accordance with the invention.

### DETAILED DESCRIPTION

Specific embodiments of the invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the in the context of treating vascular lesions, the invention may also be used for lesions or calcium deposits in other body passageways where it is deemed useful. Furthermore, there is no intention to be bound by any expressed or implied theory presented in the preceding technical field, background, brief summary or the following detailed description.

The invention is directed to methods and devices for localized delivery of one or more calcium chelating agents within the cardiovascular system in order to remove calcium from an atherosclerotic plaque lesion or a cardiac valve. Calcification is a biomineralization process in which calcium phosphate is deposited within or on tissues. Chelating agents are organic compounds that bind ionized calcium (Ca⁺⁺) in aqueous solutions, including bodily fluids. Once bound, the calcium is sequestered and is no longer available to interact with phosphate, proteins, lipids and other substances. The bound calcium is then excreted without further reaction within the body. Calcium chelating agents have a high equilibrium constant and deplete the soluble Ca⁺⁺ in a fluid volume. If a calcium chelating agent is administered to the blood volume surrounding a calcified plaque lesion, Ca⁺⁺ with the accompanying phosphate ion is solubilized into the serum and removed from the plaque lesion. Synthetic and naturally derived calcium chelating agents may be utilized in the invention.

Synthetic and naturally derived calcium chelating agents that can be used within the body include, but are not limited to, 2,2′-bipyridyl, dimercaptopropanol, ionophores, ethylenediaminotetraacetic acid (EDTA), ethylene glycol-bis-(2-aminoethyl)-N,N,N′,N′-tetraacetic acid (EGTA), nitrilotriacetic acid, NTA ortho-phenanthroline, gramicidin, monensin, valinomycin, salicylic acid, triethanolamine (TEA), polysaccharides, organic acids with at least two coordination groups such as citric acid and acetic acid, lipids, steroids, amino acids, peptides, phosphates, phosphonates, nucleotides, tetrapyrrols, ferrioxamines, and phenolics. In one embodiment, the calcium binding agent is used singly. In other embodiments, calcium binding agents are used in combination to produce the desired decalcification of the plaque lesion or cardiac valve. In one embodiment, local delivery of one or more calcium chelating agents allows the therapeutic decalcification of plaque without generalized depletion of calcium stores from the bones and/or other parts of the body.

In other embodiments, agents such as chemoattractants for osteoclasts or compounds that switch cell phenotype towards osteoclast development are delivered alone or with calcium chelating agents to the treatment site. Various cytokines, vascular endothelial growth factor, and other appropriate compounds may be delivered to the vessel surrounding the calcified plaque to recruit osteoclasts to the treatment site. The increased population of osteoclasts stimulates calcium mobilization from the treatment site and therefore stimulates demineralization of the calcified plaque. In one embodiment, agents convert monocytes and macrophages residing in the plaque to active osteoclasts, which are capable of breaking, absorbing, and/or remodeling the calcium depositions within the plaque or at the treatment site. Exemplary agents for osteoclast therapy include, but are not limited to, M-CSF (macrophage-colony stimulating factor), RANKL (receptor activator of nuclear factor-κ ligand), IL-6 (Interleukin 6), Prostaglandin E2, and the like.

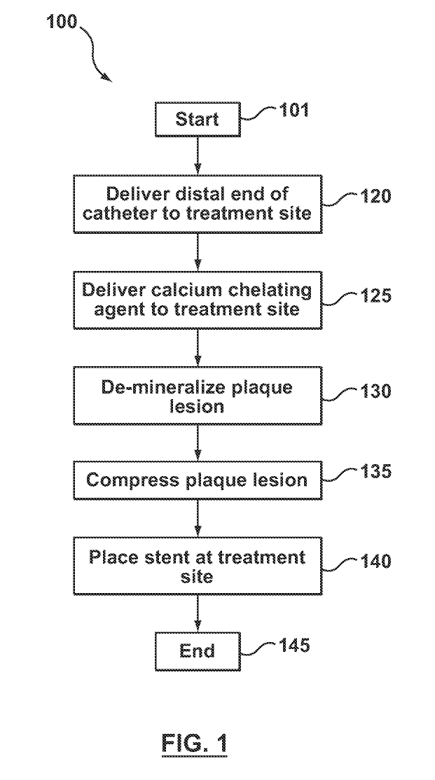

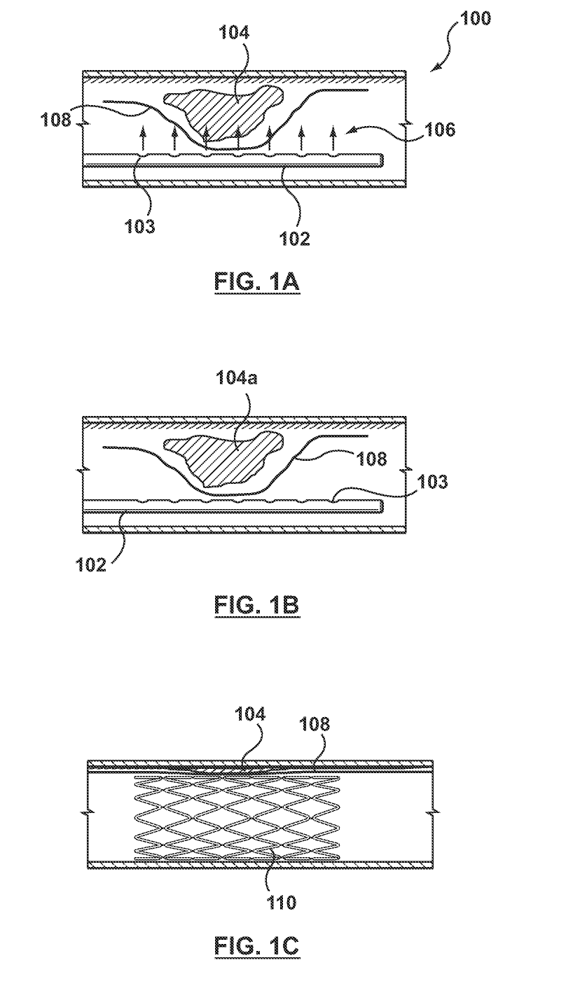

Referring now to FIGS. 1 to 1C, FIG. 1 is a flow chart of one embodiment of a method 150 for treating a calcified plaque lesion and FIGS. 1A to 1C are schematic illustrations of the method 100 for treating calcified plaque lesions shown in FIG. 1. Method 100 begins at step 101. At step 120 a delivery catheter for delivering the calcium chelating agent is inserted and advanced to the treatment site having the calcified plaque lesion. As shown in FIG. 1A, a delivery catheter is inserted into the vascular system and the distal portion 102 of the catheter is positioned adjacent to calcified plaque lesion 104. One or more calcium chelating agents are released through at least one opening 103 in the wall of distal portion 102, (step 125). The calcium chelating agent is released in the vicinity of calcified plaque lesion 104, as indicated by de- arrows 106. With the release of the calcium chelating agent, the de-mineralization of the calcified plaque lesion 104 begins, (step 130). The calcium chelating agents pass through collagen layer 108 and bind Ca⁺⁺ surrounding lesion 104. As the concentration of Ca⁺⁺ in the aqueous fluid surrounding plaque lesion 104 decreases, the calcium phosphate within plaque lesion 104 dissolves. The newly released Ca⁺⁺ is also bound by the chelating agent, causing the decalcification process to continue. Due to the de-mineralization process, the calcium content of plaque lesion 104a decreases, as indicated by the lighter shade of gray in FIG. 1B. The de-mineralized plaque lesion 104a, also defined herein as a pliable plaque, consists largely of lipid, cellular debris and inflammatory cells, and is soft and pliable.

In one embodiment of method 100, the de-mineralized plaque lesion is compressed against the wall of the vessel, (step 135). Compression may be accomplished by performing an angioplasty procedure or directly stenting the lesion. If an angioplasty procedure is performed, the angioplasty balloon is used to compress the plaque against the vessel wall. Compression of the plaque against the vessel wall restores or otherwise increases blood flow through the vessel. Preferably, compression of the de-mineralized plaque is accomplished without cracking the plaque and damaging the vessel wall. In one embodiment, a distal protection device is positioned downstream of the compression device to capture any emboli that may be released due to the compression of the lesion.

As shown in FIG. 1C, treatment and compression can be performed by placing stent 110 across lesion 104, (step 140). In one embodiment, stent 110 is delivered and expanded at the treatment site subsequent to a compression of the lesion by an angioplasty balloon. In another embodiment, stent 110 is used as the only compression device for treating the de-mineralized lesion. When stent 110 is expanded against the vessel wall, plaque lesion 104 is compressed or further compressed resulting in restored and/or increased blood flow and within the vessel. After removal of all catheters, method 100 ends at 145.

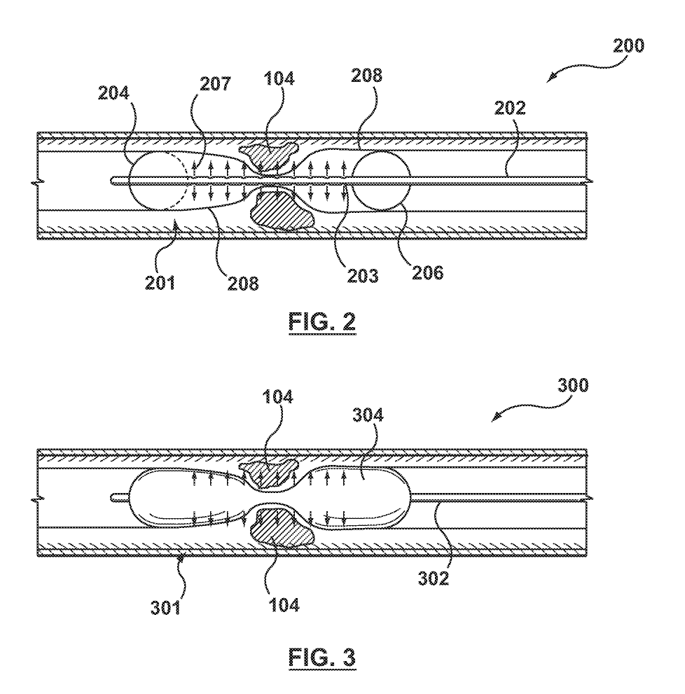

In accordance with the present invention, calcium chelating agents are delivered to the treatment site through catheters of varying design, depending on the size and location of the calcified plaque. FIG. 2 illustrates a dual balloon catheter system 200 for treating a calcified plaque lesion by delivering a calcium chelating agent locally to a calcium-rich vascular lesion. In one embodiment, dual balloon catheter system 200 is used for treating a calcified plaque lesion in accordance with method 100. In this embodiment, two inflatable balloons 204 and 206 are mounted on a distal portion 201 of catheter 202. In one embodiment, catheter 202 is inserted into the cardiovascular system and the distal portion 201 of catheter 202 is advanced to the treatment site. Catheter distal portion 201 is positioned so that calcified plaque lesion 104 is located between distal balloon 204 and proximal balloon 206. Proximal balloon 206 is inflated against vascular wall 208, stopping blood flow to the treatment site, and allowing blood to drain distally from the segment of the vessel surrounding plaque lesion 104. Next, distal balloon 204 is inflated forming a sealed chamber with vascular wall 208, between balloons 204 and 206, surrounding plaque lesion 104. A solution of one or more calcium chelating agents is then released from catheter 202 through a plurality of openings 203 within the wall of into the chamber, as indicated by arrows 207 in FIG. 2. The sealed chamber confines the calcium chelating agent to the area surrounding plaque lesion 104 for a period of time that is adequate to allow decalcification of plaque lesion 104. In one embodiment, the time period for decalcification is between five minutes and one hour depending on the size and surface area of calcified plaque lesion 104. When decalcification of plaque lesion 104 is sufficient, distal balloon 204 is deflated, and the decalcifying agent is washed away. Next, proximal balloon 206 is deflated, blood flow is allowed to resume, and catheter 202 is withdrawn from the body. In another embodiment, catheter 202 includes an internal lumen that allows blood to flow through the catheter during treatment and maintain blood flow during the course of the intervention.

FIG. 3 illustrates another embodiment of a catheter system 300 for treating a calcified plaque lesion. Catheter system 300 includes a compliant delivery device for conforming to the geometry of a vessel and delivering a calcium chelating agent locally to a calcium-rich vascular lesion. Catheter system 300 is used to deliver a calcium chelating agent directly to calcified plaque lesion 104. A single expandable, highly compliant balloon 304 is mounted on the distal portion 301 of catheter 302. When positioned across calcified plaque lesion 104 and expanded, balloon 304 surrounds and envelopes plaque lesion 104. In one embodiment, balloon 304 is porous and one or more chelating agents are dissolved in a fluid medium and expressed through the pores of the balloon directly onto the surface of plaque lesion 104. In another embodiment, a perfusion lumen through the length of the balloon is used to maintain blood flow during treatment of plaque lesion 104.

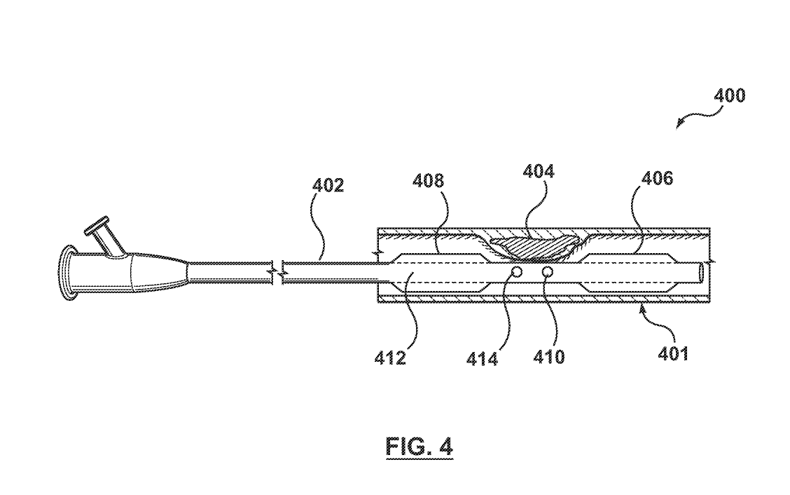

FIG. 4 is illustrates another embodiment of a catheter system 400 for treating a calcified plaque lesion. Catheter system 400 comprises a dual balloon catheter for delivering a calcium chelating agent locally and for withdrawing the calcium chelating agent at the end of the treatment. In this embodiment, the dual balloon catheter is used for forming a sealed chamber around a calcified plaque and allowing the chelation fluid to be withdrawn through the catheter at the end of the treatment. Catheter system 400 includes a distal balloon 406 and a proximal balloon 408 mounted on a distal portion 401 of catheter body 402. The distal portion 401 of catheter 402 is positioned so that the calcified plaque lesion 404 is between balloons 406 and 408 to treat the lesion. Proximal balloon 408 is inflated, stopping blood flow to the treatment site, and allowing blood to drain distally from the segment of the vessel surrounding the plaque lesion. Next, distal balloon 406 is inflated, and a sealed chamber is formed within the vessel between balloons 406 and 408, surrounding the plaque lesion 404. A solution of one or more calcium chelating agents is then injected from a lumen 412 within catheter 402 through port 410 in catheter 402 into the chamber, to treat the lesion. At the end of the treatment period, the chelation fluid is withdrawn from the sealed chamber between balloons 406 and 408 through suction port 414 back into the lumen 412 of catheter 402 and removed from the body, thus reducing the exposure of other bodily tissues to the chelating agent. In one embodiment, an aqueous fluid is injected through port 410 while fluid is withdrawn through suction port 414 so that the chelating fluid is flushed from the sealed chamber without drawing a vacuum on the vessel wall and potentially harming the tissues of the vessel wall.

One aspect of the invention includes administering a particulate chelating agent into the vessel wall in proximity to the calcified lesion. The particulate chelating agent can be in the form of microparticles, nanoparticles, or nanocrystals of the chelating agent; or microspheres or nanospheres containing or bound to one or more chelating agents. In one embodiment, the chelating agent comprises nanocrystaline ethylenediaminotetra-acetic acid (EDTA) or ethylene glycol-bis-(2-aminoethyl)-N,N,N′,N′-tetraacetic acid (EGTA). In another embodiment, one or more chelating agents such as organic acids (citric acid, acetic acid, or another weak acid, for example), lipids or steroids are bound to microspheres having a diameter less than 10 microns. The microospheres comprise one or more of a variety of biocompatible materials such as polylactic acid and its copolymers, polyamide esters, polyvinyl esters, polyvinyl alcohol, polyanhydrides, natural biodegradable polymers, such as polysaccharides, liposomes, vesicles, and any other appropriate material. These materials may be used alone or in various combinations to give the microspheres unique properties such as controlled rates of degradation, and to provide the desired rate of delivery of the chelating agent.

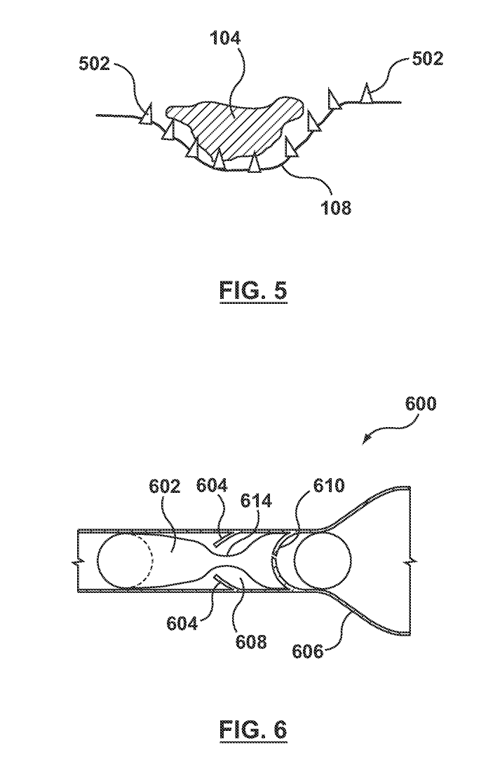

In one embodiment, the particulate chelating agent is suspended in a delivery fluid, such as dimethyl sulfoxide (DMSO), propylene glycol, or the like, and delivered to the treatment site using a dual balloon catheter such as those described above for systems 300 or 400, shown in FIG. 3 and FIG. 4, respectively. At the treatment site, a sealed chamber is formed between the balloons, and the delivery fluid is injected or otherwise released into the chamber. As shown in FIG. 5, chelating agent particles 502 migrate through collagen layer 108 overlaying plaque lesion 104. Particulate chelating agent 502 is then released at the site of calcified plaque lesion 104 as the particles dissolve or are eluted from the microspheres.

In one embodiment, two calcium chelating agents are delivered to the treatment site. A first calcium chelating agent may be either in solution or a suspension of particles in a delivery medium capable of penetrating collagen layer 108. A second calcium chelating agent comprises a solution that acts primarily on the surface of calcified plaque. When the two chelating agents are injected into a sealed chamber surrounding the calcified plaque to be treated, the second chelating agent acts primarily on the surface of the plaque while the chamber is maintained, and then is either washed away when blood flow resumes, or is withdrawn through a suction port into the catheter at the end of the treatment. The first chelating agent enters the vessel wall by penetrating the collagen layer overlaying the calcified plaque and remains at the treatment site for a period of time following treatment. In an embodiment where the first chelating agent is a liquid, it would remain at the site of the calcified plaque for several minutes, but would act on surface area not accessible from the lumen of the vessel. In an embodiment where the first chelating agent is particulate, the chelating agent is released over a period of days or weeks, and thus extends the time and extent of decalcification of the plaque lesion.

In yet another embodiment, a chelating agent is dissolved in a hydrogel and coated on the luminal surface of the vessel over the calcified plaque lesion. The hydrogel may consist of hydrophilic polymers such as polyethylene oxide, polyhydroxyl methacrylate, polyvinyl alcohol, and other suitable polymers. These polymers may be combined with biodegradable polymers such as lactide, caprolactone, trimethylene carbonate, caprolactone derivatives, and glycolides. The polymers are used in combinations to provide a hydrogel that degrades and is removed from the treatment site within a defined period of time. In one embodiment, the hydrogel degrades within a time period of one day to one week. The chelating agent is dissolved or suspended in the hydrogel and applied to the luminal wall of the vessel at the site of the calcified plaque using a catheter with a compliant, porous balloon, such as device 300, shown in FIG. 3. After the catheter is withdrawn, the hydrogel remains as a coating on the vessel wall and releases the chelating agent at the treatment site for a defined period of time. After release of the chelating agent is complete, the hydrogel degrades and is removed from the treatment site.

FIG. 6 illustrates a device 600 for de-mineralizing a calcified heart valve. In this embodiment, device 600 comprises a stent graft having a valve member on the luminal surface. The stent graft is contoured so that it forms a sealed chamber when expanded within a cardiac chamber. Stent graft 600 comprises an expandable tubular body 602. In the expanded configuration, a central portion of tubular body 602 has a diameter that is smaller than the end portions, giving tubular body 602 an hour glass shape. Stent graft 600 is sized to be temporarily positioned across calcified heart valve 604 and expanded against inner wall 606 of the cardiac chamber so that cardiac valve 604 is placed within sealed chamber 608. An artificial heart valve 610 is attached to the surface of the inner lumen of tubular body 602, and functions in place of native valve 604 during treatment by maintaining unidirectional blood flow through the lumen of stent graft body 602. Outer surface 614 of tubular body 602 is coated with one or more calcium chelating agents that are released into sealed chamber 608. In one embodiment, the calcium chelating agent is nano-particulate and enters the cardiac chamber wall where it provides a depot of chelating agent that is released over a period of time in proximity to the heart valve.

In one embodiment, device 600 is positioned across cardiac valve 604 using a catheter. Tubular body 602 is expanded against inner wall 606 of the cardiac chamber, forming sealed chamber 608 around valve 604. Tubular body 602 is left in place while one or more calcium chelating agents are released from outer surface 614 of tubular body 602 into sealed chamber 608. The calcium chelating agents are maintained in the area surrounding valve 604 for a period of time that is adequate to allow decalcification of valve 604. During the treatment period, blood flow is maintained through the central lumen of tubular body 602. At the end of the treatment period, device 600 is removed from the body, and the decalcified valve 604 resumes its function.

While the invention has been described with reference to particular embodiments, it will be understood by one skilled in the art that variations and modifications may be made in form and detail without departing from the spirit and scope of the invention.

1. A method for treating a plaque lesion comprising:
delivering into a vascular wall at a treatment site at least one calcium chelating agent; and
releasing a therapeutically effective amount of the calcium chelating agent to the treatment site for a preselected period of time;
wherein the calcium chelating agent removes calcium from mineralized portions of the plaque lesion causing the plaque lesion to become pliable plaque.

2. The method of claim 1 further comprising positioning a stent across the pliable plaque at the vascular wall.

3. The method of claim 1 further comprising positioning the pliable plaque circumferentially against the vessel wall with a balloon.

4. The method of claim 1 wherein a first chelating agent is delivered into a vascular wall at the treatment site and a second chelating agent is delivered to a luminal surface of the vascular wall at the treatment site.

5. The method of claim 4 wherein the first chelating agent comprises microparticles.

6. The method of claim 4 further comprising removing the second calcium chelating agent from the treatment site via suction ports on a catheter.

7. The method of claim 1 wherein the calcium chelating agent is selected from the group consisting of 2,2′-bipyridyl, dimercaptopropanol, ethylenediaminotetraacetic acid (EDTA), ethylene glycol-bis-(2-aminoethyl)-N,N,N′,N′-tetraacetic acid (EGTA), ionophores, nitrilotriacetic acid, NTA ortho-phenanthroline, gramicidin, monensin, valinomycin, salicylic acid, triethanolamine (TEA), polysaccharides, organic acids with at least two coordination groups, lipids, steroids, amino acids, peptides, phosphates, phosphonates, nucleotides, tetrapyrrols, ferrioxamines, and phenolics.

8. The method of claim 1 wherein the calcium chelating agent is a microparticulate.

9. A device for treating a calcified heart valve comprising:
a tubular graft member having a luminal surface and an outer surface; and
a valve member attached to the luminal surface, wherein when placed in a blood vessel at a treatment site, the outer surface of the graft member forms a sealed chamber adjacent the vessel wall at the treatment site.

10. The method of claim 9 wherein the outer surface is coated with a calcium chelating agent.

11. A method of treating a calcified heart valve comprising:
delivering a tubular graft member to a treatment site adjacent the calcified heart valve;
placing the tubular graft member through the calcified heart valve;
expanding the tubular graft member against the vessel wall to form a sealed chamber that encloses the calcified heart valve;
delivering at least one chelating agent into the sealed chamber to remove calcium from the heart valve.

12. The method of claim 11 wherein a first chelating agent is delivered into a cardiac chamber wall adjacent to the heart valve, and a second chelating agent is delivered to into the sealed chamber that encloses the heart valve.

13. The method of claim 12 wherein the first chelating agent comprises microparticles.

14. A system for treating a plaque lesion comprising:
means for delivering into a vascular wall at a treatment site at least one calcium chelating agent; and
means for releasing a therapeutically effective amount of the calcium chelating agent to the treatment site for a preselected period of time;
wherein the calcium chelating agent removes calcium from mineralized portions of the plaque lesion causing the plaque lesion to become pliable plaque.

15. A method for treating a plaque lesion comprising:
delivering into a vascular wall at a treatment site at least one agent that recruits osteoclasts to the treatment site; and
releasing an effective amount of the agent at the treatment site for a preselected period of time;
wherein the agent increases osteoclast population at the treatment site and stimulates calcium removal from mineralized portions of the plaque lesion causing the plaque lesion to become pliable plaque.

16. The method of claim 15 wherein the agent is selected from the group consisting of M-CSF (macrophage-colony stimulating factor), RANKL (receptor activator of nuclear factor-κ ligand), IL-6 (Interleukin 6), and Prostaglandin E2.

17. A method for treating a plaque lesion comprising:
delivering into a vascular wall at a treatment site at least one agent that converts monocytes and macrophages residing in the plaque lesion to active osteoclasts; and
releasing an effective amount of the agent at the treatment site for a preselected period of time;
wherein the active osteoclasts alter calcium depositions within a location selected from the group consisting of the plaque lesion and the treatment site.

18. The method of claim 17 wherein the active osteoclasts alter calcium depositions by a mechanism selected from breaking, absorbing, and remodeling the calcium depositions.

19. The method of claim 17 wherein the agent is selected from the group consisting of M-CSF (macrophage-colony stimulating factor), RANKL (receptor activator of nuclear factor-κ ligand), IL-6 (Interleukin 6), and Prostaglandin E2.
